(19) United States
(12) Patent Application Publication
(10) Pub. No.: US 2005/0214370 A1
(43) Pub. Date: Sep. 29, 2005

(54) Stable coating agent comprising sterol

(76) Inventors: Robert Joseph Sarama, Loveland, OH (US); Raymond Louis Niehoff, Hamilton, OH (US); Wayne Edward Beimesch, Taylor Mill, KY (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/811,256

(22) Filed: Mar. 26, 2004

(51) Int. Cl. A61K 31/56; A61K 9/48; A61K 9/20; A61K 9/24

(52) U.S. Cl. 424/472; 514/169

(57) ABSTRACT

An ingestible coating agent comprising: (a) sterol, and (b) solvent. The coating agent can be used to protect ingestible substrates from adverse conditions that would otherwise lead to degradation of the substrate. Preferably, the solvent comprises azeotropic solvent. In another aspect, the present invention provides a coated substrate comprising an ingestible coating and an ingestible substrate. The coating agent can be used to coat any suitable substrate. Suitable substrates can include, but are not limited to, vitamins, amino acids, minerals, phytochemicals, carotenoids, pharmaceuticals, salts, nutrients, physiological active agents, and mixtures thereof.

### FIELD OF THE INVENTION

The present invention is directed to stable coating agents, especially stable coating agents comprising sterol. The coating agents are ingestible and can be used to coat ingestible substrates, such as nutrients and pharmaceuticals.

### BACKGROUND OF THE INVENTION

Many consumers desire more nutritious food products. In response to this demand, the food industry has developed products that contain added nutrients, such as vitamins, in an effort to provide foods with added health benefits. However, the processes used to prepare many of these food products can expose the nutrients to conditions that adversely affect nutrient integrity.

Many vitamins and many other substrates used as nutritional additives are highly labile and thus incapable of withstanding these processes. These substrates commonly react or degrade when exposed to conditions such as ambient light, excessive acid or base, moisture, heat, oxygen, or the presence of chemically incompatible substrates. As a result of these reactions, the substrates can completely or partially lose their nutritional value and thus become unable to provide the desired health benefits.

For example, it is desirable to add nutrients to various foods. However, when such foods are subjected to heated conditions via cooking (e.g., flying), the nutrients can experience thermal decomposition and/or oxidation, leading to significant vitamin loss.

Many attempts have been made in the art to maintain the integrity of nutritional substrates by using a coating material to form a protective barrier between the substrate and the adverse condition. Some approaches have included spraying cellulose derivatives and lipid material onto the nutrient, or admixing the nutrient with liquid carriers having a polarity opposite of that of the nutrient. Other approaches have used high speed disk processing to contact the nutrients with a molten matrix to form beadlets. Still other approaches have involved techniques such as compressing the nutrient with compressible tablet forming material or applying a protective film to a nutrient caplet. See, e.g., U.S. Pat. Nos. 4,182,778; 4,943,437; 5,008,118; 6,432,448 B1; and 6,555,145 B1.

Prior coatings have commonly been directed towards protecting against mechanical pressure, moisture, and storage conditions. However, these attempts have not been wholly satisfactory. For example, such coatings have not been capable of protecting nutrients from excessive processing temperatures. Furthermore, once coated, the nutrients have not been released from the coating agent at an appropriate time after ingestion to provide full bioavailability. In many prior applications, nutrients are released in the mouth or stomach, leading to premature degradation, nutrient to nutrient interaction, or taste problems. In other applications, the nutrient is released beyond the ideal intestinal point of maximum absorption.

Thus, it would be desirable to provide a coating that protects ingestible substrates, particularly nutrients such as vitamins, from heat and other environmental influences. It would also be desirable to provide an embodiment of such a coating that releases the nutrients in the part of the digestive system where they are highly bioavailable.

This and other objects of the invention will become apparent from the following disclosure.

### SUMMARY OF THE INVENTION

The present development provides an ingestible coating agent comprising: (a) sterol, and (b) solvent. The coating agent can be used to protect ingestible substrates from adverse conditions that would otherwise lead to degradation of the substrate. Furthermore, the coating agent breaks down in the bile salts of the small intestine, thus allowing substrates, such as vitamins, to become available for absorption at a point in the digestive system where they are highly bioavailable.

Preferably, the solvent comprises azeotropic solvent in one embodiment, an azeotropic solvent has a Hildebrand Solubility Index of from about 8.2 to about 9.2. In another embodiment, the azeotropic solvent has a Snyder Polarity Index of from about 1.0 to about 2.1.

In a particular embodiment, the sterol comprises: (a) stigmasterol, and (b) sterol having a melting point of from about 40 degrees Celsius to about 170 degrees Celsius.

In another aspect, the present invention provides a coated substrate comprising an ingestible coating and an ingestible substrate. In one embodiment, the ingestible coating has a thermal resistance of from about 100 degrees Celsius to about 170 degrees Celsius. In another embodiment, the ingestible coating has a pH resistance of from about 0.1 to about 10. In yet another embodiment, the ingestible coating has an oxidative resistance value of from about 95% to about 100%. And in yet another embodiment, the ingestible coating has a Water Solubility Index of from about 0% to about 1%.

The coating agent can be used to coat any suitable substrate. Suitable substrates can include, but are not limited to, vitamins, amino acids, minerals, phytochemicals, carotenoids, pharmaceuticals, salts, nutrients, physiological active agents, and mixtures thereof.

### DETAILED DESCRIPTION OF THE INVENTION

A. Definitions

As used herein, “azeotropic” means a solvent mixture having a vapor-liquid composition that remains essentially constant through the point of drying.

As used herein, “sterol” means one or a combination of two or more sterols. As used herein, the term “sterol” includes sterols, stanols (the ring-saturated derivatives of sterols), and mixtures thereof.

As used herein, “solvent” means one or a combination of two or more solvents.

As used herein, “food” means food or beverage product.

These definitions are intended to apply throughout this application unless a different meaning is plainly specified.

All percentages are by weight unless otherwise specified.

All documents cited herein are, in relevant part, incorporated by reference; the citation of any document is not to be construed as an admission that it is prior art with respect to the present invention.

B. Coating Agent Components

The present invention provides an ingestible coating agent comprising: (a) sterol, and (b) solvent. In one embodiment, the coating agent comprises from about 0.001% to about 20% sterol and from about 80% to about 99.999% solvent, preferably from about 10% to about 20% sterol and from about 80% to about 90% solvent. Preferably, the ratio of sterol to solvent is greater than about 5:95, more preferably greater than about 15:85.

1. Sterol

The coating agent comprises sterol. The sterol can be one or more sterols, used either singularly or as a mixture. Any suitable sterol can be used. Examples of suitable sterols include plant sterols such as sitosterol, stigmasterol, and campesterol. Particular sterols can be selected for use singularly or in a mixture based upon the desired end properties and application. For example, sterols can be chosen based upon compositional melt point, crystallization, friability, malleability, and cohesiveness.

In one embodiment, the sterol comprises a mixture of at least two sterols. Preferably, each sterol in the mixture has a melting point of from about 40 degrees Celsius to about 170 degrees Celsius. In a particular embodiment, one of the sterols in the mixture is stigmasterol.

In another embodiment, stigmasterol and at least one other sterol, having a melting point of from about 150 degrees Celsius to about 170 degrees Celsius, are used. In this embodiment, enhanced thermal resistance is desired, thus the sterols chosen have high melting points relative to the substrate to be coated and the temperature of the application. In one embodiment, a sterol that imparts a waxy texture, such as campesterol, sitosterol, or mixtures thereof, is used.

2. Solvent

The coating agent comprises solvent. In one embodiment, the preferred solvent is an azeotropic solvent. Azeotropic solvents provide maximum sterol solubilization, and have a sufficiently high vapor pressure to enable volatilization out of the sterol upon crystallization. As vaporization occurs, the azeotropic nature of the solvent is realized; this maintains the sterol in a pseudo-soluble state during the coating process and enables crystallization control during the drying process, resulting in a superior homogenous coating.

Although any suitable solvent can be used, preferred solvents have a Hildebrand Solubility Index of from about 8.2 to about 9.2, and a Snyder Polarity Index of from about 1.0 to about 2.1. Furthermore, the solvent is preferably non-chlorinated. Additionally, the solvent is preferably in a single phase both alone and with the sterol added.

In one embodiment, the solvent comprises an initial mixture of 70% hexane and 30% ethanol, resulting in an azeotropic molar ratio of 66.8:33.2, respectively, during subsequent drying. In another embodiment, the initial solvent comprises 8% ethyl acetate and 92% ethanol, resulting in an azeotropic molar ratio of 54:46, respectively, during subsequent drying. This particular solvent has a Hildebrand Solubility Index of about 8.7 and a Snyder Polarity Index of about 1.6.

3. Optional Ingredients

The coating agent of the present invention may additionally comprise any suitable optional ingredients such as, but not limited to, excipients such as disintegrants, colorants, opaquants, flavorants, or combinations thereof.

Disintegrants can include any suitable disintegrants such as, but not limited to, croscarmellose sodium, starch, starch derivatives, clays, gums, cellulose, cellulose derivatives, alginates, crosslinked polyvinypyrrolidone, sodium starch glycolate, microcrystalline cellulose, calcium carbonate, or pectin.

The preferred coating agent as described herein will normally only dissolve when exposed to the bile salts in the small intestine, thus releasing the substrate into the digestive tract. Disintegrants can be included in the coating agent if it is desired to have the substrate released earlier, such as in the stomach. Therefore, when the coated substrate arrives in the stomach, the disintegrants can cause the coating agent to breakdown and thus release the substrate. Preferably, if disintegrants are added, they constitute less than about 10% of the coating agent. In one embodiment, the disintegrants are not wetted or solublized by the sterol or solvent; rather, they are provided as a suspension in the coating agent. In a particular embodiment of the invention, calcium carbonate is added to the coating agent, causing fracturing of the sterol coating under the influence of gastric fluids or stomach acid. This results in release of the substrate prior to entry into the small intestine.

The coating agent herein may include colorants or opaquants to alter or change the color of the coated substrate. Any suitable colorant may be used such as, but not limited to, titanium dioxide, food dyes, lakes, natural vegetable colorants, iron oxides, silicates, sulfates, magnesium hydroxide, and aluminum hydroxide. Preferably, if colorants are added, they comprise less than about 0.01% of the coating agent.

The coating agent described herein can also optionally comprise flavorant to contribute to or enhance the flavor of the coated substrate. These can include natural and synthetic flavors and mixtures thereof, such as but not limited to spray-dried flavors. Preferably, if flavorants are added, they comprise about 0.05% or less of the coating agent.

C. Method for Making the Coating Agent

The present invention also relates to a preferred method for making the coating agent. The method comprises mixing the sterol or sterol mixture with solvent to form the coating agent.

The sterol and the solvent are combined by dissolving the sterol in the solvent. In one embodiment the coating agent comprises from about 0.001% to about 20% sterol and from about 80% to about 99.999% solvent, preferably from about 10% to about 20% sterol and from about 80% to about 90% percent solvent. In one embodiment the ratio of sterol to solvent is greater than about 5:95, preferably greater than about 15:85.

Only about 3% of the sterols typically dissolve if a solvent with a Hildebrand Solubility Index of from about 8.2 to about 9.2 and a Snyder Polarity Index of from about 1.0 to about 2.1 is not used. However, using a solvent having a Hildebrand Solubility Index of from about 8.2 to about 9.2 and a Snyder Polarity Index of from about 1.0 to about 2.1 allows for greater dissolution of sterol to achieve maximum dissolution of from about 15% to about 20% of the sterol in the solvent. The mixture of sterol and solvent, combined as the coating agent, can be heated to just below the solvent's boiling temperature. This temperature is preferably maintained during the coating process, providing additional sterol solubilization.

D. Coated Ingestible Substrate

The current invention is also directed to a coated substrate, which is an especially preferred use of the coating agent. The coated substrate comprises: (a) the coating agent; and (b) an ingestible substrate. The ingestible substrate can be in any suitable form, such as a solid or a liquid in a matrix. Thus, the size and shape of the substrate are relevant only as they relate to the particular coating method employed.

Any suitable substrate can be used herein. For example, suitable substrates can include, but are not limited to, vitamins, amino acids, minerals, phytochemicals, carotenoids, pharmaceuticals, salts, nutrients, physiological active agents, and mixtures thereof.

Substrates can include any nutrient. Nutrients can include, but are not limited to, vitamin B₁, vitamin B₂, vitamin B₆, vitamin B₁₃, vitamin B₁₄, vitamin B₁₅, lipoic acid, nicotinic acid, nicotinamide, pantothenic acid, folic acid, p-aminobenzoic acid, biotin, choline, inositol, vitamin C, and mixtures thereof; or a calcium, sodium, potassium, magnesium, iron, copper or zinc salt derivative of the aforementioned nutrients. Substrates can also include a water-soluble derivative of oil-soluble nutrients, such as vitamin A, vitamin D, vitamin E, or vitamin K, and other nutrients rendered water-soluble by derivatives, and mixtures thereof.

Substrates can also include, but are not limited to, enzymes, amino acids, peptides, polypeptides, polypeptide hormones, phytochemicals, carotenoids, minerals, salts, and combinations thereof. Amino acids can include, but are not limited to, α-amino acids, β-amino acids, other amino acids, peptides, or mixtures thereof. Polypeptides can include insulin. Substrates can also include calcium, sodium, potassium, magnesium, iron, copper or zinc salts, other salts such as hydrochrolates or nitrates of the aforementioned amino acids, derivatives such as esters of phosphoric acid or acetic acid, salts formed by two or more kinds of the aforementioned amino acids, and mixtures thereof.

Phytochemicals can include, but are not limited to, allyl sulfides, indoles, glucosinolates, sulfaforaphane, phthalides, silymarin, monoterpenes (e.g., limonene), ellagic acid, phenols, flavonoids (e.g., quercetin, isoflavones), polyacetylenes, isothiocyanates, thiocyanates, phytic acid, saponins, glycyrrhizin, catechins, thiols, omannoheptulose, and mixtures thereof. Carotenoids can include, but are not limited to, lycopene, beta-carotene, cyptoxanthin, zeaxanthin, and mixtures thereof.

Minerals can include, but are not limited to, calcium, magnesium, manganese, boron, chromium, cobalt, copper, iron, molybdenum, selenium, silicon, zinc, and mixtures thereof. Salts can include, but are not limited to, those containing fluorine, iodine, chlorine, or mixtures thereof.

Substrates can also include any suitable pharmaceutical.

E. Method for Making the Coated Substrate

Any suitable method can be used for making the coated substrate, including spray and pan coating. A preferred method for coating the substrate with the coating agent uses a Lakso Wurster™ Fluid Bed Coater (herein referred to as the “Wurster”).

In a preferred method of using the Wurster, the substrate to be coated is greater than about 150 microns in size. The feed tube and spray nozzle are adjusted for the particular application. The Wurster is then loaded with substrate. The air inlet flow is started and the temperature is set to about 125 degrees Fahrenheit (52 degrees Celsius) or less than the boiling point of the solvent. The Wurster is then warmed up and the atomizing air is set to about 130 degrees Fahrenheit (54 degrees Celsius) and about 20 psi (138 kPa). The fluidizing airflow rate through the Wurster is controlled to from about 20 standard cubic feet per minute (SCFM) to about 40 SCFM (about 9.4 L/sec to about 18.9 L/sec). The coating agent is then applied to the base particle in the Wurster at a flow rate of about 12 grams/minute (0.2 grams/second) to about 18 grams/minute (0.3 grams/second). This process is continued until the desired amount of coating agent is applied. The level of coating applied is dependent on the porosity of the substrate and the intended application of the coated substrate. Hot air flow is continued until the exit gas temperature is greater than about 110 degrees Fahrenheit (43 degrees Celsius) to evaporate any residual solvent. The coated substrate is then recovered from the Wurster and sieved. It should be noted that one of ordinary skill in the art can manipulate the equipment variables and settings, some examples being the inlet/outlet temperatures, air flow rate, and rate at which coating agent is applied, so as to achieve the desired coating. It is important to note that the use of azeotropic solvent helps to provide a coating that is contiguous and essentially uniform without defects.

F. Properties of the Coated Substrate

The coating can provide a barrier to chemical reactants, and can be resistant to both water and lipids found in food formulations. This benefit is useful, for example, in food products where a particular substrate like a vitamin is desired as an additive. Coating the vitamin can allow the food product to undergo increased temperatures without losing nutritional value. In addition, the coating agent can form a coat that is capable of providing water impermeability such that unwanted dissolution, hydration, or hydrolysis does not occur when the vitamin is exposed to certain conditions, and oxidative resistance such that oxygen labile nutrients are able to withstand exposure to oxygen. Also, the coating can be oil insoluble, thus preventing lipid soluble nutrients from escaping into the lipid phase where oxidation of the nutrients would proceed more rapidly.

In one embodiment, the coating has a thermal resistance of from about 100 degrees Celsius to about 170 degrees Celsius. In another embodiment, the coating has a pH resistance of from about 0.1 to about 10. In yet another embodiment, the coating has an oxidative resistance value of from about 95% to about 100%. And in yet another embodiment, coating has a Water Solubility Index of from about 0% to about 1%.

Another advantage of using the coating agent can be protection and control of unwanted reactions between formulation components when in an aqueous phase. In one embodiment of the invention, calcium is coated for use in a beverage that contains linear polyphosphate (SHMP) for treatment of dental erosion. In general, SHMP binds with calcium ions. Therefore, without controlling the calcium-SHMP binding action, no SHMP can be available for dental erosion control. By coating the calcium source with the coating agent and making it available as a small or nano-particle, the calcium-SHMP binding action can be prevented and the calcium can be suspended in the beverage with minimal settling and with minimal aesthetic negatives.

In another embodiment, beta-carotene is coated and used in pet foods, which undergo processing conditions that normally would degrade the beta-carotene. Once coated, the highly volatile beta-carotene does not interact with water or oxygen. Also, the taste of the coated substrate is controlled because the coating is fairly malleable and hence is not fractionable on impact. Thus, the unpleasant flavor and color of the beta-carotene is contained.

G. Analytical Methods

1. Thermal Resistance

Thermal resistance involves evaluating the coating melt point, using the visible melt-point (optical microscopy with hot stage) method as known in the art. The sample is put on the microscope, ramped up at 10 degrees Celsius per minute, and the on-set of the coating's melting is determined by looking for liquid pooling or a shiny glaze appearing on the substrate.

2. Oxidative Resistance

In oxidatively labile substances, this general approach is used to measure the oxidative stability of the coated substrate. While the actual analytical method will be specific to the particular substrate coated, the test method involves the following. Assay the level of oxidative labile active in the substrate prior to coating. Remeasure the level of active substrate after coating while accounting for the increased mass of the coating. Note: losses of active during coating are to be controlled to a minimum. Establish the level of active in the coated substrate. Determine the oxidative resistance of the coated substrate by subjecting the coated substrate to ambient temperature and air for 12 months. Remeasure the level of active in the coated substrate.
Oxidative Resistance Value:

$$Formula = \frac{\begin{matrix}( mg . active in coated substrate at time zero ) - \\ ( mg . active in coated substrate at 12 months )\end{matrix}}{( mg . active in coated substrate at time zero )} * 100$$

3. pH Resistance

This parameter is determined in accordance with ASTM E70-97(2002), “Standard Test Method for pH of Aqueous Solutions with the Glass Electrode.”

4. Water Solubility Index

Water solubility index (WSI) is measured using a modified method of Anderson, et al., 1969, Gelatinization of Corn Grits by Roll and Extrusion Cooking, Cereal Sci. Today 14:4-12. Two grams of sample are mixed with 25 ml of water and put into a centrifuge tube. The sample mixture is heated for 30 minutes in a water bath at 30 degrees Celsius and then mixed for 5 minutes using a vortex mixer. The mixture is then centrifuged at 3,000 RPM for 10 minutes. The WSI is determined using the following equation:
WSI%=(Weight of dissolved solid in supernatant/weight of dry sample solids in original sample)×100

5. Hildebrand Solubility Index

This parameter is measured in accordance with the method set forth in U.S. Pat. No. 5,120,369.

6. Snyder Polarity Index

This parameter is determined in accordance with “Classification of the Solvent Properties of Common Liquids,” L. R. Snyder, J. Chromatogr., 92, 223 (1974); J. Chromatogr. Sci., 16, 223 (1978).

### H. EXAMPLES

The present invention is illustrated by the following non-limiting examples:

### Example 1

### Coating 10% Beta-Carotene Beadlet in a Lakso™ Wurster Fluidized Bed Coater

A riser tube of 7 inches (18 cm) is installed with a 10 mm gap. Load the base material and set the air flow to 30 SCFM (14.2 L/sec) and inlet temperature raised to 120° F. (49° C.). Dissolve 15% sterol in 85% solvent by weight and heat this mixture (the coating agent) and maintain during the coating at 130° F. (54° C.). This solvent forms an azeotropic mixture during drying at 66.8% hexane and 33.2% ethanol molar mixture. The spray nozzle and assembly are preheated to 120° F. 49° C.). Atomizing pressure is 20 psi (138 kPa) using a 20/50/70 nozzle. The flow rate of the coating agent is 13 grams/minute (0.22 g/sec). Outlet temperature during the application is 105° F. (41° C.). Continue process applying all of the coating. Continue drying after all coating material is applied. Turn off the heat to the fluidizing air when the outlet temperature rises to 110° F. (43° C.). Continue air flow until outlet temperature drops to 105° F. (41° C.). Remove coated material and sieve to remove fines and agglomerates as needed.

| | |
|---|---|
| 500 grams | Beta Carotene, BASF ® (sieved to remove fines) |
| | (BASF Corp., Mt. Olive, New Jersey, USA) |
| 500 grams | Phytosterol* mixture, ADM ® (ADM, Decatur, |
| | Illinois, USA) |
| 870 grams | Ethanol 200 proof, Aaper ® (Aaper Alcohol, |
| | Shelbyville, Kentucky, USA) |
| 2030 grams | Hexane, J. T. Baker ® (J.T. Baker Chemical Co., |
| | Phillipsburg, New Jersey, USA) |

(*Phytosterols: Melting Point 140° C., Brassicasterol 3.2%, Campesterol 11.1%, Stigmasterol 16.1%, B-Sitosterol 44.0%, Sitostanol 2.1%, Waxes 7.7%.)

### Example 2

### Coating a Vitamin Mix (Nutrients A, D, and K) with 33.3% Stigmasterol and 66.6% Sisterol

A operational conditions are the same as in Example 1. In this example, 17% sterol mixture is dissolved in the solvent.
500 grams Vitamin mix, BASF® (BASF Corp., Mt. Olive, N.J., USA)
100 grams Sitosterol, Sigma® (Sigma Chemical Co., St. Louis, Mo., USA)
50 grams Stiginasterol, Sigma® (Sigma Chemical Co., St. Louis, Mo., USA)
596 grams Hexane J. T. Baker® (J. T. Baker Chemical Co., Phiilipsburg, N.J., USA)
261 grams Ethanol 200 proof, Aaper® (Aaper Alcohol, Shelbyville, Ky., USA)

1. A coating agent comprising:
(a) sterol;
(b) solvent.

2. The coating agent of claim 1, wherein said solvent comprises an azeotropic solvent.

3. The coating agent of claim 2, wherein said azeotropic solvent has a Hildebrand Solubility Index of from about 8.2 to about 9.2.

4. The coating agent of 3, wherein said azeotropic solvent has a Snyder Polarity Index of from about 1.0 to about 2.1

5. The coating agent of claim 4, wherein said azeotropic solvent comprises hexane and ethanol.

6. The coating agent of claim 4, wherein said azeotropic solvent comprises ethyl acetate and ethanol.

7. The coating agent of claim 1, wherein said sterol comprises:
(a) stigmasterol; and
(b) sterol having a melting point of from about 40 degrees Celsius to about 170 degrees Celsius.

8. The coating agent of claim 4, wherein said sterol comprises:
(a) stigmasterol; and
(b) sterol having a melting point of from about 40 degrees Celsius to about 170 degrees Celsius.

9. The coating agent of claim 7, comprising:
(a) from about 0.05 wt. % to about 20 wt. % sterol; and
(b) from about 80 wt. % to about 99.95 wt. % solvent.

10. The coating agent of claim 9, comprising:
(a) from about 10 wt. % to about 15 wt. % sterol; and
(b) from about 85 wt. % to about 90 wt. % solvent.

11. A coated substrate comprising:
(a) the coating agent of claim 1; and
(b) an ingestible substrate.

12. A coated substrate comprising:
(a) the coating agent of claim 4; and
(b) an ingestible substrate.

13. A coated substrate comprising:
(a) the coating agent of claim 8; and
(b) an ingestible substrate.

14. The coated substrate of claim 11, wherein said ingestible substrate is selected from the group consisting of vitamins, amino acids, minerals, phytochemicals, carotenoids, pharmaceuticals, salts, nutrients, physiological active agents, and mixtures thereof.

15. The coated substrate of claim 13, wherein said coated substrate has a thermal resistance of from about 100 degrees Celsius to about 170 degrees Celsius.

16. The coated substrate of claim 15, wherein said coated substrate has a pH resistance of from about 0.1 to about 10.

17. The coated substrate of claim 16, wherein said coated substrate has an oxidative resistance value of from about 95% to about 100%.

18. The coated substrate of claim 17, wherein said coated substrate has a Water Solubility Index (WSI) of from about 0% to about 1%.

19. A coated substrate comprising:
(a) an ingestible coating, wherein said ingestible coating has:
(1) a thermal resistance of from about 100 degrees Celsius to about 170 degrees Celsius;
(2) optionally, a pH resistance of from about 0.1 to about 10;
(3) optionally, an oxidative resistance value of from about 95% to about 100%;
(4) optionally, a Water Solubility Index of from about 0% to about 1%; and
(b) an ingestible substrate.

20. A coated substrate comprising:
(a) an ingestible coating, wherein said ingestible coating has:
(1) a thermal resistance of from about 100 degrees Celsius to about 170 degrees Celsius;
(2) a pH resistance of from about 0.1 to about 10;
(3) an oxidative resistance value of from about 95% to about 100%;
(4) a Water Solubility Index of from about 0% to about 1%; and
(b) an ingestible substrate.
